(19) United States
(12) Patent Application Publication
(10) Pub. No.: US 2022/0265984 A1
(43) Pub. Date: Aug. 25, 2022

(54) INTERLOCKING SAFETY MEDICAL CONNECTORS WITH PROTECTIVE LOCKING CAPS

(71) Applicants: Michael W. Howlett, Las Vegas, NV (US); James V. Mercer, West Jordan, UT (US); Brad C. Holt, Midvale, UT (US)

(72) Inventors: Michael W. Howlett, Las Vegas, NV (US); James V. Mercer, West Jordan, UT (US); Brad C. Holt, Midvale, UT (US)

(21) Appl. No.: 17/675,992

(22) Filed: Feb. 18, 2022

Related U.S. Application Data
(60) Provisional application No. 63/151,186, filed on Feb. 19, 2021.

(51) Int. Cl. A61M 39/10; A61M 5/162

(52) U.S. Cl. CPC A61M 39/1011; A61M 5/162; A61M 2039/1077; A61M 2039/1072

(57) ABSTRACT

An interlocking medical connector configured to linearly drive a deliver needle perpendicularly through one or more septa by using a rotational moment to advance the needle without twisting. A non-rotatable junction and spinning collar that drives the male and female connectors together without needle twisting. A locking mechanism that requires an intentional motion by a user to disconnect, thereby reducing the risk of inadvertent disconnection. Additional absorbent layers to capture fluid that may escape the fluid pathway. And corresponding protective locking connector caps with an absorbent component that isolates, absorbs, and deactivates hazardous fluid residues on the connectors.

### CROSS-REFERENCE TO RELATED APPLICATIONS

This application claims priority to and the benefit of U.S. Provisional Patent Application No. 63/151,186, filed on Feb. 19, 2021, which is hereby incorporated by reference in its entirety.

### FIELD OF THE INVENTION

The interlocking safety medical connectors with protective locking caps fall within the field of single use medical connectors used for administering IV fluids and medications to include hazardous drugs.

### BACKGROUND

The need for specialized safety connectors for hazardous drug (HD) administration has long been established. To date, many of these closed system transfer devices (CSTD) utilize a method that linearly pushes male and female connectors together, thus driving a needle through two septa in a membrane-to-membrane connection, used to secure and isolate the medication within the system, thus opening a fluid pathway. These devices have latches and other similar mechanisms to keep the two connectors secured together during use. However, many clinical reports pertaining to these connectors indicate that these connectors become inadvertently disconnected during therapy resulting in unwanted hazardous drug exposures, calling into question the reliability of the devices currently on the market.

Almost all standard medical connectors utilize a clockwise twist-on application similar to a Luer lock or compatible fittings. Twist-on connectors provide the advantages that they are, intuitive, the rotational torque used in conjunction with a screw-type ramp provides greater linear force than simply pressing two connectors together, and they are more secure in that the connection is less likely to come apart when screwed together.

To date, there are no HD membrane-to-membrane connectors that utilize the tried and proven method of a screw or twist-on union. Part of the reason for this is that needles penetrating a septum cannot be safely rotated or twisted as the needle enters the septum. This twisting may result in the needle damaging the septum. This damage could include the generation of small particles of polymer that could contaminate the medication and enter the patient's blood stream via the vascular access device, although the more common outcome would be septal damage that leads to leakage of the potentially hazardous drug. Further, current connectors do not prevent a slight variation in the preferred perpendicular needle angle as it enters the septum. This oblique needle entry also increases risk of septal damage and improper creation of a fluid pathway.

HD medications can provide lifesaving treatment for an affected patient, but these same medications can put the health care providers at risk following long term exposure to even microscopic amounts of the drugs. There is a common misconception in HD therapy that the current connectors have a completely “dry” disconnect with some manufacturers claiming there is no residual medication on the connectors after use. This is inaccurate. There is always at least a microscopic amount of fluid on any connector after use and disconnection.

What is needed is an HD medical connector that has the increased linear force to drive the delivery needle perpendicularly through one or more septa without rotation of the needle as it passes through the septa, provides the intuitive and secure connection of a twist-on fitting and makes provisions to contain and control any residual medications left on the HD connector through absorption and neutralization and by capping the disparate components after disconnect.

### BRIEF SUMMARY AND OBJECTS OF THE INVENTION

The disclosure of the present invention relates to an INTERLOCKING SAFETY MEDICAL CONNECTOR or more specifically to a two-part HD medical connector configured to deliver hazardous medications to a patient in a safe and secure manner.

The device of the present invention solves the problems of easily driving a delivery needle perpendicularly through one or more septa by using a novel screw-drive method to advance the needle without the needle twisting. This is accomplished by providing a non-rotatable junction and utilizing a spinning “nut” or collar that drives the male and female connectors together perpendicularly without needle twisting.

Additionally, the device of the present invention provides other safeguards to prevent hazardous fluid exposure. First, by adding an encasing layer of absorbent material proximate to the septum or septa, such as a foam ring or outer foam layer on the septa, second, by incorporating a fluid wiping (e.g., “squeegee”) mechanism to further isolate HD within an air gap formed between the septum and the fluid wiping layer, and lastly, by providing capping mechanisms which twist onto the male and female sides of the HD connector and will absorb and neutralize the residual hazardous fluid. These caps also protect the connector from touch contamination and may also be used to disinfect the connectors with an antiseptic substance.

A first embodiment of the interlocking safety medical connector or, HD connector, including a two-part male and female connector, the male side configured to be attached to an intravenous delivery system such as an IV or needless injection site (NIS) fitted to the patient, the female side is the delivery side and configured to be attached via IV tubing to, such as, an IV bag, syringe or medical pump, the female side connector is the side closet to the medical practitioner and is considered the delivery side connector.

The male side connector including an outlet, a fixed collar, a coupling shoulder and an anti-twist post; a fluid delivery pathway is formed along the central axis of the male side connector having continuity through the connector. The outlet of the present invention configured as a male Luer or similar device having a twist on connection for IV tubing. The fixed collar is formed around the outlet portion and allows the user to easily manipulate the connector when making the junction with the patient side IV tubing or with the corresponding female side of the HD connector. The fixed collar having facets, grooves, or ridges in order to provide secure handing. The coupling shoulder and anti-twist post are formed following the fixed collar. The coupling shoulder having a pair of helical grooves or lock pin ramps originating at the free end of the coupling shoulder and terminating proximate the base of the coupling shoulder with a recessed opening or lock pin capture area, the lock pin capture area is rectangular in shape and extends away from the base of the lock pin ramp. The height of the lock pin capture area is substantially larger than the diameter of the reciprocal lock pins. The upper edge of the capture area including a protrusion extending downward between the base of the lock pin ramp and the close end of the upper edge, the retainer protrusion configured to prevent the lock pin from entering the base of the lock pin ramp and the connectors becoming detached without precise manipulation by the user. The anti-twist post is configured to be inserted into a reciprocal receiver formed in the female side connector, the post including at least one facet or keyed face in order to prevent rotation within the receiver. The anti-twist post of one embodiment of the present invention includes six equidistant facets, or is formed as a hexagon, with the receiver of the female side connector being shaped as a hexagon of is similar but slightly larger size. It is recognized that the anti-twist post can be formed as any number of geometric shapes, irregular shapes or even a simple oval in order to prevent twisting within the reciprocal receiver.

At least one septum is installed proximate to the inlet side of the fluid pathway formed through the male side connector.

The female or delivery side connector, including in part, an inlet cap, an inlet fitting, a delivery needle, a spring housing, a spring, a needle alignment sleeve, a spinning lock collar, a spring cap with anti-twist receiver and at least one septum.

The inlet cap including the inlet fitting and the delivery needle. The inlet cap attaches to the spring housing as a snap-on feature, and the inlet cap is free rotating independent of the spring housing. This will allow the HD connector to spin or rotate and prevents an attached IV tubing from kinking or twisting during installation or actual use. The inlet fitting on the inlet cap configured as a twist-on IV tubing coupling or male Luer fitting, the tubing coupling having a helical tread configured to retain standard IV tubing. In other embodiments, the tubing coupling might be a coupling having one or more spurs or may be a twist on fitting having a reciprocal fitting which is already coupled with an IV tubing. The inlet having a fluid pathway in communication with a delivery needle. The delivery needle is configured as a “non-coring” needle with a sealed point and a discharge opening in the side of the needle proximate the point; the edges of the discharge opening are chamfered or smoothed over in order to avoid the delivery needle cutting away particles of the septa or absorbent sponges that may be penetrated in the device. The delivery needle being fixed at the terminating end of the fluid pathway and will pass fluid through the remaining components of the female side connector through the septum in the male side connector and into the patient side IV distribution mechanism.

The spring housing configured to house a biasing element such as a bias spring, a needle alignment sleeve and a spring cap with the anti-twist receiver, the spinning lock collar is configured to fit over the spring housing at the end distal from the inlet fitting. A needle pathway is formed through the needle alignment sleeve with at least one septa fitted into the needle pathway proximate the distal end of the alignment sleeve.

When the male and female side connectors of the present invention are fitted together, the user will align the connectors into an approximately linear position with the end of the anti-twist post of the male connector sitting in the reciprocal opening formed in the spring cap with anti-twist receiver. The user will twist the spinning lock collar in a clockwise motion with slight to modest pressure holding the male and female connectors together, traveler pins or lock pins on the inside of the spinning lock collar will engage the opening of the reciprocal helical lock pin ramps or grooves formed in the shoulder of the male side connector, the twisting motion will move the lock pins down the ramp and into the pin capture area. As the lock pins are moved down the ramp the two sides of the HD connector are driven together, pushing the delivery needle through the first septum fixed in the needle alignment sleeve and then through the second septum fixed in the fluid pathway formed through the anti-twist post, allowing for fluid to be safely passed through the HD connector. Once the lock pins are in the pin capture area pressure from the bias spring will force the lock pins against the edge of the capture area and into the root of the retainer protrusion. In order for the HD connector to be disconnected, the user must use two hands to grasp the male side connector and the female side connector, force the two sides together while rotating the spinning lock collar in a counter-clockwise manner in order to move the lock pins below the retainer protrusion and into the base of the locking ramps. This functionality is similar to a safety cap for many medicine bottles and requires a degree of training for medical personnel and clear intentional actions by the medical personnel in order to separate the HD connector of the present invention. This will prevent inadvertent disconnection.

In one embodiment of the present invention, it is contemplated that an additional absorption sponge, configured as a round washer with a central opening, is placed within the base of the spinning lock collar in order to control and absorb HD medication drips created when disconnecting the male and female sides of the connector. In a first embodiment the sponge may be treated using an antiseptic, such as isopropyl alcohol (IPA) in order to neutralize any biological elements in the medication or in second embodiment the sponge may contain a variety of materials, such as activated charcoal, that will act to both neutralize and absorb any residual hazardous elements. In one embodiment the sponge may be placed over the alignment post and aide in biasing the locking pins against the top edge of the pin capture area.

In a first embodiment the septa or septum of the present invention may be formed as a simple plug or membrane used preserve a sterile environment within the spring housing and protect the delivery needle and used to contain any HD medication which is residual within the IV delivery system. The septa must be formed using a sterile elastomeric material with properties to allow the delivery needle to penetrate and then functionally re-seal once the delivery needle is withdrawn.

In another embodiment the septum is formed as two layers, where a first layer is a self-sealing elastomeric material and the second layer is a sponge layer. The elastomeric layer configured to contain HD medications to a “fluid delivery side” of the delivery system and the sponge used to wick up and isolate any trace amount of the HD medication that is pulled through the septum by the delivery needle.

In yet another embodiment, the septum can be formed having an elastomeric core configured to allow the delivery needle to pass through and to seal once the needle is retracted, and outside layer of sponge configured to absorb or wick up any residual medication that is deposited on the face of the septum. In another embodiment, the face of the septum will include a radial pattern of micro grooves designed to more readily transport even small amounts of HD fluid to the perimeter sponge material. The sponge in each of the above described embodiments may contain materials, such as activated charcoal, that facilitates neutralization a particular HD medication.

The septum of another embodiment may include more than one layer separated by a sealed air gap. This may be a septum layer formed using an elastomeric material followed by a more rigid layer configured as a fluid wiping layer, and again each of the layers separated by an air gap. If HD fluid is carried through septum on the delivery needle it would be wiped clean by the fluid wiping layer, depositing the HD fluid into the sealed air gap. In a first embodiment the fluid wiping layer is pierced by the delivery needle, in a second embodiment it is envisioned that the fluid wiping layer would include a slit that allows the delivery needle to pass through but the slit having a sufficient restriction in order to wipe any liquid traces away from the delivery needle.

### BRIEF DESCRIPTION OF THE DRAWINGS

The following description of the embodiments can be understood in light of the Figures, which illustrate specific aspects of the embodiments and are part of the specification. Together with the following description, the Figures demonstrate and explain the principles of the embodiments.

FIG. 1 an exploded perspective of the interlocking safety medical connector,

FIG. 2 a perspective of the male and female side connectors,

FIG. 3 a perspective of the male connector,

FIG. 4 a perspective of the female side connector with reciprocal cap,

FIG. 5 a perspective of the male side connector with reciprocal cap,

FIG. 6A a perspective of the female side cap,

FIG. 6B a perspective if the male side cap,

FIGS. 7A and 7B are perspectives of septa of the present invention,

FIG. 8 is a perspective of an absorbent sponge, and,

FIGS. 9A and 9B are top sectional elevations of a septum.

### DETAILED DESCRIPTION OF THE ILLUSTRATED EMBODIMENTS

The following description of the embodiments can be understood in light of the Figures which illustrate specific aspects of the embodiments and are part of the specification. Together with the following description, the Figures demonstrate and explain the principles of the embodiments. In the Figures the physical dimensions of the embodiment may be exaggerated for clarity. The same reference numerals or word descriptions in different drawings represent the same element, and thus their descriptions may be omitted.

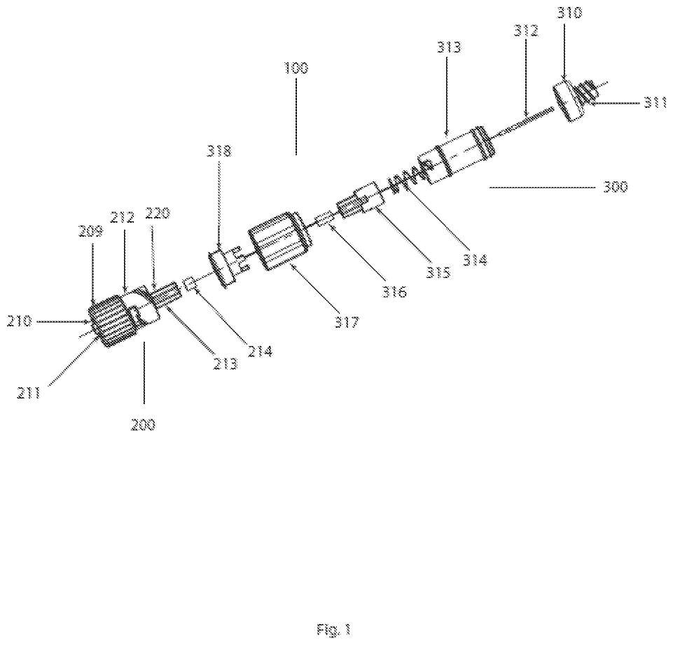

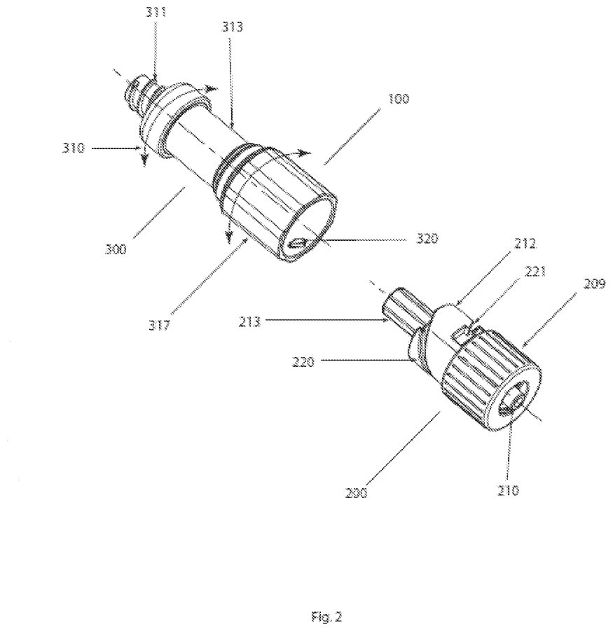

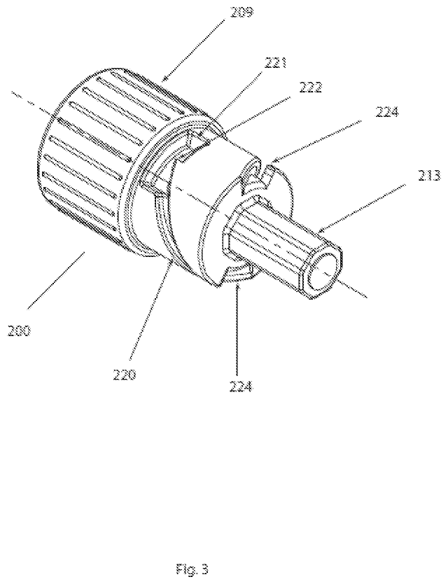

A first embodiment of the present invention or Interlocking Safety Medical Connector 100, as described in FIGS. 1 through 3 including a male side connector 200 and a female side connector 300.

The male side connector 200 or distal side connector including an outlet 210, a fixed collar 209, a coupling shoulder 212 and a keying features, such as an anti-twist post 213 having a plurality of longitudinal facets; a fluid delivery pathway is formed along the central axis of the male side connector 200 having continuity through the connector. The outlet 210 configured as a male Luer 211 or similar device having a twist on connection for IV tubing. The fixed collar 209 is formed around the outlet portion 210 and allows the user to easily manipulate the male side connector 200 when making the junction with the patient side IV tubing or with the corresponding female side 300 of the HD connector 100. The fixed collar 209 having, facets, grooves, or ridges in order to provide secure handing. The coupling shoulder 212 and anti-twist post 213 are formed following the fixed collar 209. The coupling shoulder 212 having a pair of helical grooves or lock pin ramps 220 originating at the free end of the coupling shoulder 212 forming a ramp opening 224 and the lock pin ramps 220 terminating proximate the base of the coupling shoulder 212 with a recessed opening or lock pin capture area 221, the lock pin capture area is rectangular in shape and extends away from the base of the lock pin ramp 220. The height of the lock pin capture area 221 is at least two times the diameter of the reciprocal lock pins 320 formed on the inside of the spinning lock collar 317 of the female side connector 300. The upper edge of capture area 221 including a protrusion extending downward between the base of the lock pin ramp and the close end of the upper edge, the retainer protrusion 222 configured to prevent the lock pin 320 from entering the base of the lock pin ramp 220 and the connectors 200 and 300 becoming detached without precise manipulation by the user. The anti-twist post 213 is configured to be inserted into a reciprocal receiver 318 formed in the female side connector 300, the post 213 including at least one facet or keyed face in order to prevent rotation within the receiver 318. The anti-twist post 213 of one embodiment of the present invention includes six equidistant facets or is formed as a hexagon with the receiver 318 of the female side connector 300 being shaped as a hexagon of is similar but slightly larger size. It is recognized that the anti-twist post 213 can be formed as any number of geometric shapes, irregular shapes or even a simple oval in order to prevent twisting within the reciprocal receiver 318. At least one septum 214 is installed proximate the inlet side of the fluid pathway formed through the male side connector 200. Other keying features can include complementary post and groove or other longitudinally slidable interfaces that limit and/or prevent rotational movement relative to one another.

The female side connector 300 including, in part, an inlet cap 310, an inlet fitting 311, a delivery needle 312, a spring housing 313, a spring 314, a needle alignment sleeve 315, a spinning lock collar 317, a spring cap with anti-twist receiver 318 and at least one septum 316.

The inlet cap 310 including the inlet fitting 311 and the delivery needle 312. The inlet cap 310 attaches to the spring housing 313 as a snap on feature, and the inlet cap 310 is free rotating independent of the spring housing 313. The inlet fitting 311 on the inlet cap 310 configured as a twist-on IV hose coupling like a male Luer, the hose coupling having a helical tread configured to retain standard IV hose. In other embodiments, the hose coupling might be a coupling having one or more spurs or may be a twist on fitting having a reciprocal fitting which is already coupled with an IV hose. The inlet 311 having a fluid pathway in communication with a delivery needle 312. The delivery needle 312 configured as a “non-coring” needle with a sealed point and a discharge opening in the side of the needle proximate the point; the edges of the discharge opening are chamfered or smoothed over in order to avoid the delivery needle 312 cutting away particles of the septa or absorbent sponges that may be penetrated in the device. The delivery needle 312 being fixed at the terminating end of the fluid pathway and will pass fluid through the remaining components of the female side connector 300 through the septum 214 in the male side connector 200 and into the patient side IV distribution mechanism.

Some embodiments include a biasing element to bias a needle alignment sleeve longitudinally away from a housing of the biasing element. In the illustrated embodiment, the biasing element is a spring, and the housing is a spring housing. The spring housing 313 configured to house a bias spring 314, a needle alignment sleeve 315 and a spring cap with the anti-twist receiver 318, the spinning lock collar 317 is configured to fit over the spring housing 313 at the end distal from the inlet fitting. A needle pathway is formed through the needle alignment sleeve 315 with at least one septa 316 fitted into the needle pathway proximate the distal end of the alignment sleeve 315.

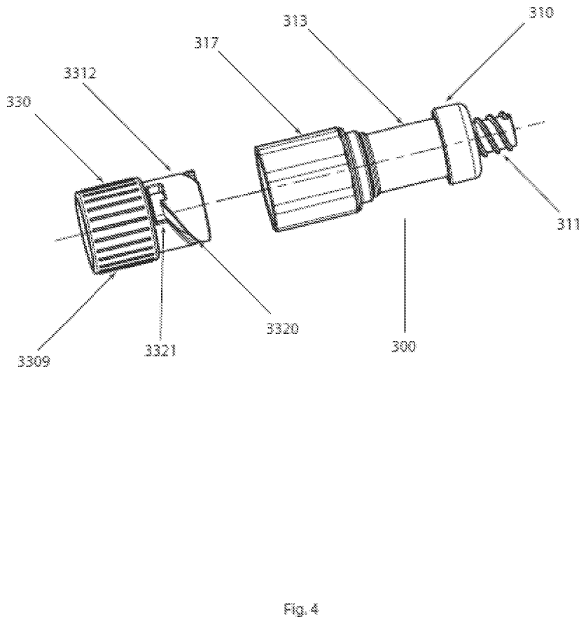

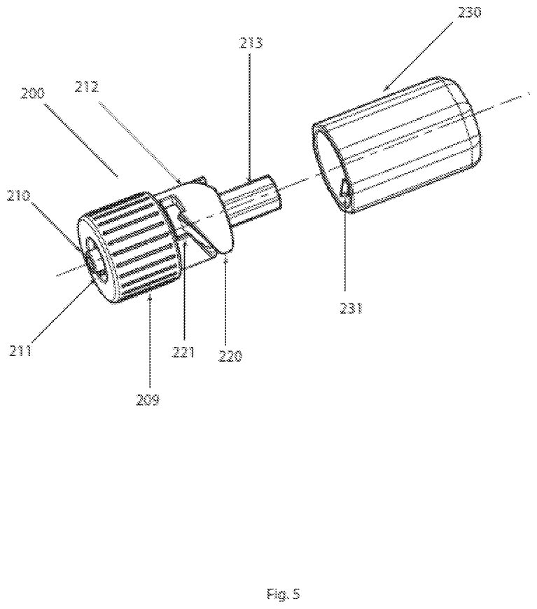

Each of FIGS. 4 and 5 are embodiments of caps for the female side connector 300 and the male side connector 200, respectively.

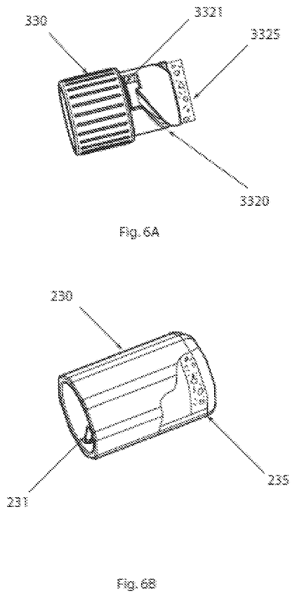

FIG. 4 showing the female side connector 300 having an inlet cap 310 with inlet fitting 311 on the proximal end of spring housing 313 and spinning lock collar 317 on the distal end. The female connector cap 330 configured having a ribbed collar 3309 and a cap shoulder 3312. A cap lock pin ramp 3320 formed in the side of the cap shoulder 3312 with a similar configuration as described above for lock pin ramp 220 of the male side connector 200 including a lock pin capture portion 3321. It is contemplated that a foam sponge 3325 (FIG. 6A) is fixed to the end of cap 330. The sponge 3325 may include an antiseptic, such as, IPA or may include an absorbent or neutralizing material such as activated carbon. The sponge 3325 will also act to bias lock pins 320 of spinning lock collar 317 into the cap pin capture area 3321.

FIG. 5 depicts the male side connector 200 having the elements as described above including a coupling shoulder 212 having helical lock pin ramps 220, with the pin capture area 221 and the anti-twist post 213. The male side connector cap 230 configured to cover alignment post 213 and fit over the coupling shoulder 212 when the alignment pin 231 are moved down the lock pin capture ramps 220 and into the pin capture area 221. The male side connector cap 230 may include a sponge or semi-resilient foam insert 235 (FIG. 6B) placed on the inside of cap 230. The sponge is configured to bias connector pins 231 into the pin capture area 221 when the cap is installed. The sponge may also include an antiseptic or neutralizing agent.

It is contemplated that an additional absorption sponge 500, configured as a round washer with a central opening 511, is placed within the base of the spinning lock collar 317 in order to control any HD medication drips created when disconnecting the male and female sides of the connector. In a first embodiment the sponge 500 may be treated using an antiseptic, such as isopropyl alcohol (IPA) in order to neutralize any biological elements in the medication or in second embodiment the sponge may contain activated carbon used to absorb and neutralize any isotopes used in a chemo treatment. In one embodiment the sponge 500 may be placed over the alignment post 213 and aide in biasing the locking pins 320 against the top edge of the pin capture area 221.

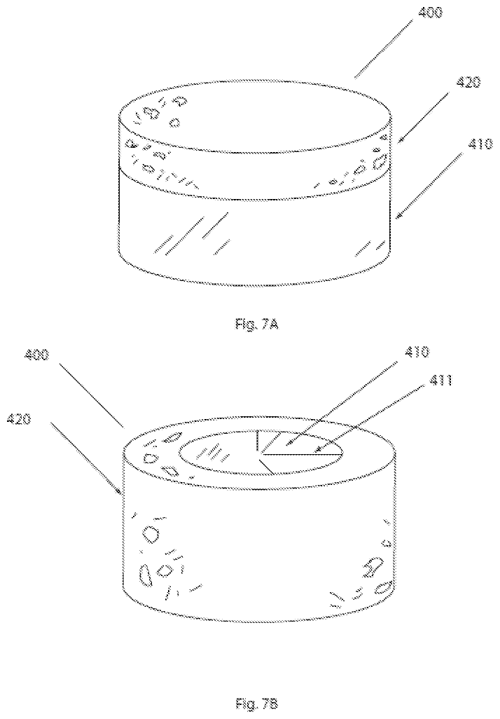

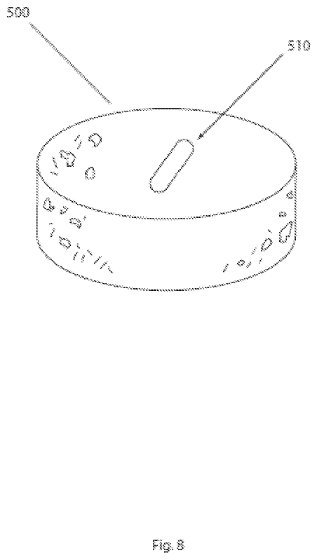

In FIG. 7A the septa or septum 400 may be formed as a simple plug or membrane used preserve a sterile environment within the spring housing 313 and protect the delivery needle 312 and is used to contain any HD medication which is residual within the IV delivery system. The septa 400 must be formed using a sterile elastomeric material with a proper durometer to allow the delivery needle to penetrate and then functionally re-seal once the delivery needle is withdrawn. In a second embodiment the septum 400 is formed as two layers, where a first layer is a self-sealing elastomeric material 410 and the second layer is a sponge layer 420. The elastomeric layer 410 configured to contain HD medications to a “fluid entry side” of the delivery system and the sponge 420 used to wick up and isolate any trace amount of the HD medication that is pulled through the septum 400 by the delivery needle 312.

In yet another embodiment shown in FIG. 7B the septum 400 can be formed having an elastomeric core 410 configured to allow the delivery needle 312 to pass through and to seal once the needle 312 is retracted, an outside layer of sponge 420 configured to wick up and residual medication that is deposited on the face of the septum 400. In another embodiment, the face of the septum elastomeric core 410 will include a radial pattern of micro grooves 411 designed to more readily transport even small amounts of HD fluid to the perimeter sponge material 420. The sponge 420 each of the above described embodiment may be treated to neutralize a particular HD medication.

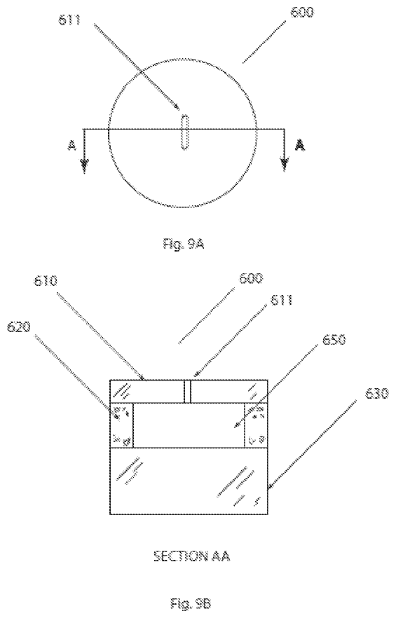

The septum 600 shown in FIGS. 9A and 9B includes more than one layer separated by a sealed air gap 650. This may be a septum layer 630 formed using an elastomeric material followed by a more rigid layer configured as a fluid wiping layer 610, and again each of the layers separated by an air gap 650. If HD fluid is carried through septum layer 630 on the delivery needle 312 it would be wiped clean by the fluid wiping layer 600, depositing the HD fluid into sealed air gap 650. In a first embodiment the fluid wiping layer 610 is pierced by the delivery needle 312, in a second embodiment it is envisioned that the fluid wiping layer 610 would include a slit 611 that allows the delivery needle 312 to pass through but having a sufficient restriction within slit 611 to wipe any liquid traces away from the delivery needle 312.

It is to be understood that the above mentioned arrangements are only illustrative of the application of the principles of the present disclosure. Numerous modifications or alternative arrangements may be devised by those skilled in the art without departing from the spirit and scope of the present disclosure and the appended claims are intended to cover such modifications and arrangements. Thus, while the present disclosure has been shown in the drawings and described above with particularity and detail, it will be apparent to those of ordinary skill in the art that numerous modifications, including, but not limited to, variations in size, materials, shape, form, function and manner of operation, assembly and use may be made without departing from the principles and concepts set forth herein.

1. An interlocking safety medical connector comprising,
a male side connector, the male side connector having:
a coupling shoulder, the coupling shoulder having at least one lock pin ramp,
an outlet,
a keying feature,
a fluid pathway formed through the coupling shoulder to the outlet, and
at least one cylindrical septum disposed in the fluid pathway; and
a female side connector, the female side connector including:
an inlet cap,
a delivery needle,
a biasing element,
a needle alignment sleeve,
a needle pathway formed through the needle alignment sleeve,
at least one cylindrical septum disposed in the needle pathway,
a spring cap with an anti-twist receiver, and
a lock collar, the lock collar having at least one lock pin configured to engage with the at least one lock pin ramp formed in the coupling shoulder of the male side connector.

2. The medical connector of claim 1 wherein the inlet and outlet caps are free rotating relative to the rest of the medical connector.

3. The medical connector of claim 1 wherein at least one septum of the septa has a elastomeric layer and an absorbent sponge layer.

4. The medical connector of claim 3 wherein the elastomeric layer is an elastomeric core, and the absorbent sponge layer is positioned around an outside diameter of the elastomeric core.

5. The medical connector of claim 4 where a face of the elastomeric core includes a radial pattern of micro-grooves.

6. The medical connector of claim 1 wherein at least one septum of the septa is formed using two layers with an air gap between the layers.

7. The medical connector of claim 6, wherein at least one of the layers is a fluid wiping layer.

8. The medical connector of claim 1 including a locking cap configured to fit the male side connector and complementarily mate with the at least one lock pin ramp.

9. The medical connector of claim 1 including a locking cap configured to fit the female side connector and complementarily mate with the at least one lock pin.

10. The medical connector of claim 1, wherein the keying feature is an anti-rotation post.

11. The medical connector of claim 10, wherein the anti-rotation post has a hexagonal cross-section and complementarily mates with a hexagonal interior cross-section of the female side connector.

12. The medical connector of claim 1, further comprising a lock pin capture area formed at the base of the at least one lock pin ramp.

13. The medical connector of claim 12, wherein the lock pin capture area includes a retainer protrusion.

14. The medical connector of claim 1, wherein the lock collar is rotatable independently of the needle alignment sleeve.

15. The medical connector of claim 1, wherein the biasing element is a spring.

16. A method of connecting a patient IV, the method comprising:
connecting the male side connector and the female side connector of claim 1, including:
inserting the at least one lock pin into the at least one lock pins ramp;
twisting the lock collar rotationally around a longitudinal axis; and
while twisting the lock collar, advancing the delivery needle longitudinally through the septa.

17. The method of claim 16, further comprising rotationally fixing the delivery needle relative to the septa while advancing the delivery needle longitudinally through the septa.

18. The method of claim 17, wherein rotationally fixing the delivery needle includes contacting the keying feature with the needle alignment sleeve.

19. The method of claim 16, further comprising penetrating a fluid wiping layer of at least one septum of the septa.

20. The method of claim 16, further comprising disconnecting the male side connector and the female side connector, including:
applying a longitudinal force to move at least a portion of the female side connector toward the male side connector; and
while applying the longitudinal force, rotating the at least one lock pin past a retainer protrusion and into the at least one lock pins ramp.
